(19) United States
(12) Patent Application Publication
(10) Pub. No.: US 2025/0064802 A1
(43) Pub. Date: Feb. 27, 2025

(54) METHODS OF TREATING IRREGULAR SLEEP-WAKE RHYTHM DISORDER AND CIRCADIAN RHYTHM SLEEP DISORDERS ASSOCIATED WITH NEURODEGENERATIVE DISEASES

(71) Applicant: Eisai R&D Management Co., Ltd., Tokyo (JP)

(72) Inventors: Margaret L MOLINE, White Plains, NY (US); Jocelyn Y CHENG, Woodcliff Lake, NJ (US); Jane YARDLEY, Hatfield (GB)

(73) Assignee: Eisai R&D Management Co., Ltd., Tokyo (JP)

(21) Appl. No.: 18/292,007

(22) PCT Filed: Jul. 20, 2022
(86) PCT No.: PCT/IB2022/056713

§ 371 Date: Jan. 25, 2024

Related U.S. Application Data
(60) Provisional application No. 63/203,512, filed on Jul. 26, 2021.

(51) Int. Cl. A61K 31/506

(52) U.S. Cl. CPC A61K 31/506

(57) ABSTRACT

Methods of treating sleep-wake rhythm disorder and related circadian rhythm sleep disorders associated with neurodegenerative diseases are disclosed.

This application claims the benefit of priority to U.S. Provisional Application No. 63/203,512 filed Jul. 26, 2021, the contents of which are incorporated herein by reference in its entirety.

Novel methods for and compositions for use in treating irregular sleep-wake rhythm disorder and related circadian rhythm sleep disorders associated with neurodegenerative diseases using lemborexant are disclosed.

Irregular Sleep-Wake Rhythm Disorder (“ISWRD”) is an internationally recognized and accepted disorder. The sleep-wake pattern exhibited by subjects with ISWRD is diagnostically distinct from insomnia. Unlike insomnia disorder, in ISWRD sleep is fragmented throughout the 24-hour day. In contrast, subjects with insomnia experience either increased sleep latency or reduced sleep maintenance at night or both, but tend not to sleep during the day. ISWRD is a disturbance of the normal circadian, or 24-hour, diurnal pattern of sleep and wake.

ISWRD is identified and coded as a distinct disorder in several International Classification systems including Circadian Rhythm Sleep Disorder, Irregular Sleep-Wake Type G47.23 (ICD-10); Circadian Rhythm Sleep Disorder, Irregular Sleep-Wake Type 307.45-3 (DSM-5); and Circadian Rhythm Sleep-Wake Disorder, Irregular Sleep-Wake Rhythm Disorder 307.45-3 (ICSD-3).

There exists an unmet medical need for a safe and effective therapy that is conveniently administered to address the irregular sleep and wake pattern, which in turn could help with problematic behaviors (e.g., sundowning, restlessness, agitation, and/or wandering) and cognitive difficulties (e.g., with memory and/or attention) associated with ISWRD.

The orexin neurotransmitter pathway is directly involved in modulation of the circadian sleep-wake rhythm, with orexins promoting wakefulness. Orexin receptors are G-protein coupled receptors found predominately in the brain. Their endogenous ligands, orexin-A and orexin-B, are expressed by neurons localized in the hypothalamus. Orexin-A is a 33 amino acid peptide and orexin-B is a 28 amino acid peptide. Sakurai, T., et al., Cell, 1998, 92, 573-85. There are two subtypes of orexin receptors: orexin receptor 1 and orexin receptor 2. Orexin receptor 1 binds orexin-A preferentially, while orexin receptor 2 binds both orexin-A and orexin-B. It has been observed that orexins control wake-sleep conditions. Chemelli, R. M., et al., Cell, 1999, 98, 437-51. Lemborexant is being studied for the treatment of insomnia disorder and for the treatment of at least one circadian rhythm sleep disorder in subjects suffering from dementia due to Alzheimer's disease (“AD”) and/or related disorders and/or mild cognitive impairment due to Alzheimer's disease. See, e.g., U.S. Patent Application Publication No. 2012/0095031 and U.S. Provisional Patent Application Nos. 62/335,599 and 62/335,611, the contents of which are herein incorporated by reference.

In some embodiments, a method of treating irregular sleep-wake rhythm disorder and related circadian rhythm sleep disorders associated with neurodegenerative diseases comprising administering to a subject in need thereof an effective amount of lemborexant, is provided. In some embodiments, prior to administration of an effective amount of lemborexant, the subject in need thereof is one who achieves a score of greater than or equal to 46 on ISWRD ObsRO Questionnaire Daytime Component. In some embodiments, prior to administration of an effective amount of lemborexant, the subject achieves a score of greater than or equal to 8 on ISWRD ObsRO Questionnaire Nighttime Component. In some embodiments, the related circadian rhythm sleep disorder is chosen from sleep disruption, sleep-wake fragmentation, and circadian rhythm sleep disorders. In some embodiments, ISWRD is due to at least one neurological disorder.

In some embodiments, a subject is being treated for ISWRD. In some embodiments, a subject is being treated for ISWRD with an effective amount of lemborexant. In some embodiments, a subject is being treated for a related circadian rhythm sleep disorder associated with neurological disorders. In some embodiments, a subject is being treated for a related circadian rhythm sleep disorder associated with neurological disorders with an effective amount of lemborexant thereof. In some embodiments, a subject experiences at least one symptom chosen from confusion, anxiety, agitation, pacing, wandering, mental exhaustion, physical exhaustion, and sundowning.

In some embodiments, neurological disorders may include neurodegenerative diseases and/or neurodevelopmental diseases.

In some embodiments, neurodegenerative diseases include but are not limited to Alzheimer's disease (AD) and other dementias, Parkinson's disease (PD) and PD-related disorders, synucleinopathies, prion disease, motor neuron diseases (MND), Huntington's disease (HD), spinocerebellar ataxia (SCA), and spinal muscular atrophy (SMA).

Neurological disorders are a large and increasing public health concern, as the population of older adults increases globally. Among the many symptoms that are associated with dementia, sleep and wake disturbances are both common and are risk factors themselves that contribute to the development and worsening of the neuropathology and symptomatology, including cognitive function and behavioral disturbances commonly referred to in the aggregate as Behavioral and Psychological Symptoms of Dementia (BPSD), based on animal models and human studies.

Sleep and wake disturbances appear early in the course of disease, e.g., Alzheimer's Disease, other dementias, etc., and are associated generally with a loss of circadian rhythmicity. Dementia subjects often have a marked decrease in sleep maintenance when they attempt to sleep during the night and can spend a significant portion of the day asleep. This pattern is referred to as sleep-wake fragmentation and is characterized by decreased amplitudes of the sleep-wake rhythms, with less predictability of the sleep-wake pattern from day to day.

As dementia progresses, subjects often exhibit behavioral disturbances such as agitation and night wandering. They often manifest disturbed behaviors that may be exacerbated in the late afternoon and early evening, in which case these are described as “sundowning.” Disrupted sleep can also increase the risk for falling, based in part on disorientation after awakening from sleep. It has been shown that subjects with the most disturbed sleep at night have more problematic behaviors during the day, for example, restlessness, agitation, and wandering. These behaviors, which also disrupt the sleep of caregivers, are reasons underlying some decisions to institutionalize dementia subjects.

There exists an unmet medical need for a safe and effective therapy that is conveniently administered to address sleep-wake fragmentation in subjects with ISWRD, which in turn would help with the problematic behaviors (e.g., sundowning, restlessness, agitation, and/or wandering) and cognitive difficulties (e.g., with memory and/or attention) associated with dementia due to Alzheimer's disease and/or other neurodegenerative disorders, and/or mild cognitive impairment due to Alzheimer's disease, and may also help to slow the brain degeneration that occurs in AD. Increased sleep duration, especially with particular sleep stages such as slow-wave sleep and REM sleep, may improve cognition. These stages are associated with performance on working memory tasks and memory consolidation, respectively. Targeting sleep may also improve behavioral disturbances in AD.

In some embodiments, a method of continuing treatment of irregular sleep-wake rhythm disorder and related circadian rhythm sleep disorders associated with neurodegenerative diseases comprising administering to a subject in need thereof an effective amount of lemborexant is provided. In some embodiments, the subject in need thereof is one who achieves a score of greater than or equal to 46 on ISWRD ObsRO Questionnaire Daytime Component. In some embodiments, the subject in need of the method of continuing treatment of irregular sleep-wake rhythm disorder and related circadian rhythm sleep disorders associated with neurodegenerative diseases achieves a score of greater than or equal to 8 on ISWRD ObsRO Questionnaire Nighttime Component. In some embodiments, the related circadian rhythm sleep disorder is chosen from sleep disruption and sleep-wake fragmentation. In some embodiments, the ISWRD is due to at least one neurodevelopmental disorder, at least one neurodegenerative disorder, or a combination thereof.

In some embodiments, after administration of an effective amount of lemborexant, the subject experiences an increase in sleep efficiency. In some embodiments, after administration of an effective amount of lemborexant, the subject experiences a decrease in daytime sleepiness and/or daytime napping. In some embodiments, after administration of an effective amount of lemborexant, the subject experiences an increase in wake efficiency. In some embodiments, after administration of an effective amount of lemborexant, the subject experiences an increase in daytime alertness. In some embodiments, after administration of an effective amount of lemborexant, the subject experiences a decrease in behavioral disturbances. In some embodiments, after administration of an effective amount of lemborexant, the subject experiences a decrease in loss in concentration. In some embodiments, after administration of an effective amount of lemborexant, the subject experiences an increase in undertaking personal care. In some embodiments, after administration of an effective amount of lemborexant, the subject experiences an increase in emotional wellbeing. In some embodiments, after administration of an effective amount of lemborexant, the subject experiences an increase in participating in leisure activities. In some embodiments, after administration of an effective amount of lemborexant, the subject experiences an increase in participating in social and occupational activities.

In some embodiments, a method of determining the therapeutic benefit in the treatment of irregular sleep-wake rhythm disorder and related circadian rhythm sleep disorders associated with neurodegenerative diseases comprising administering ISWRD ObsRO Questionnaire Daytime Component and/or ISWRD ObsRO Questionnaire Nighttime Component to a subject prior to treatment of irregular sleep-wake rhythm disorder and at least one day following treatment of irregular sleep-wake rhythm disorder is provided and comprises administering an effective amount of lemborexant.

In some embodiments, the subject to whom lemborexant is administered achieves a score of less than 46 on ISWRD ObsRO Questionnaire Daytime Component before treatment. In some embodiments, the subject to whom lemborexant is administered achieves a score of greater than or equal to 46 on ISWRD ObsRO Questionnaire Daytime Component after treatment.

In some embodiments, the subject to whom lemborexant is administered achieves a score of less than 8 on ISWRD ObsRO Questionnaire Nighttime Component before treatment. In some embodiments, the subject to whom lemborexant is administered achieves a score of greater than or equal to 8 on ISWRD ObsRO Questionnaire Nighttime Component after treatment.

In some embodiments, the methods described herein consolidate sleep during the nighttime hours. In some embodiments, the methods described herein consolidate wakefulness during the daytime hours in treated subjects. In some embodiments, after treatment, the subjects have increased wake efficiency. In some embodiments, after treatment, the subjects have increased sleep efficiency. In some embodiments, the methods described herein decrease daytime sleepiness in treated subjects. In some embodiments, after treatment, the subjects have decreased daytime sleepiness and/or daytime napping. In some embodiments, after treatment, the subjects have improved daytime alertness. In some embodiments, after treatment, subjects experience a stabilization of one or more circadian rhythms. In some embodiments, after treatment, the subjects have improvement of one or more circadian rhythms.

In some embodiments, after treatment, the subjects experience clinical stabilization in cognitive impairment. In some embodiments, after treatment, the subjects experience clinical stabilization in cognitive impairment due to a neurological disorder. In some embodiments, after treatment, the subjects experience clinical improvement in cognitive impairment due to Alzheimer's disease and/or other neurodegenerative disorders. In some embodiments, after treatment, the subjects experience clinical reduction in rate of decline in cognitive impairment due to Alzheimer's disease. In some embodiments, after treatment, the subjects experience clinical stabilization in the subject with dementia due to Alzheimer's disease and/or other neurodegenerative disorders. In some embodiments, after treatment, the subjects experience clinical improvement in the subject with dementia due to Alzheimer's disease and/or other neurodegenerative disorders. In some embodiments, after treatment, the subjects with dementia due to Alzheimer's disease and/or other neurodegenerative disorders experience clinical reduction in rate of decline. In some embodiments, the methods described herein decrease behavioral disturbances in the subject.

As used herein, the following definitions shall apply unless otherwise indicated.

As used herein, the term “lemborexant” refers to a compound having the structure:

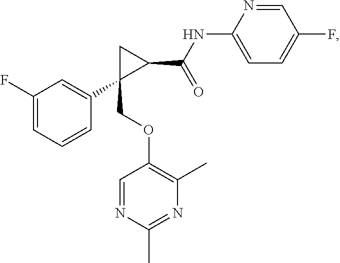 [SMILES: Cc1ncc(OC[C@@]2(c3cccc(F)c3)C[C@H]2C(=O)Nc2ccc(F)cn2)c(C)n1]

also known as (1R,2S)-2-(((2,4-dimethylpyrimidin-5-yl)oxy)methyl)-2-(3-fluorophenyl)-N-(5-fluoropyridin-2-yl)cyclopropanecarboxamide or (1R,2S)-2-(((2,4-dimethylpyrimidin-5-yl)oxy)methyl)-2-(3-fluorophenyl)-N-(5-fluoropyridin-2-yl) cyclopropane-1-carboxamide. As used herein, the term “effective amount” means an amount sufficient to effect an intended result including, but not limited to, treatment of at least one symptom associated with a disorder or condition, such as, for example, ISWRD and/or Alzheimer's disease, as illustrated below. As discussed below, in some embodiments, the effective amount in a dosage form is from 0.5 mg to 100 mg of lemborexant.

As used herein, the term “subject” means an animal subject, such as a mammalian subject, and for example, a human being. As used herein, the subject may be of any age. In some embodiments, the age of the subject may range from less than one year to more than 95 years of age. In some embodiments, the age of the subject may range from less than one year to 15 years of age. In some embodiments, the age of the subject may range from 10 years of age to 30 years of age. In some embodiments, the age of the subject may range from 25 years of age to 45 years of age. In some embodiments, the age of the subject may range from 40 years of age to 60 years of age. In some embodiments, the age of the subject may range from 55 years of age to 75 years of age. In some embodiments, the age of the subject may range from 70 years of age to 95 years of age. In some embodiments, the age of the subject may range from 60 years of age to 95 years of age.

As used herein, the terms “treatment” and “treating” refer to an approach for obtaining beneficial or desired results including, but not limited to, therapeutic benefit and/or prophylactic benefit.

As used herein, the term “ISWRD”, also known as “irregular sleep-wake pattern disorder” and “irregular sleep-wake rhythm disorder”, means a sleep cycle that is characterized in potentially having a sleep-wake pattern, however it may be accompanied by a rhythm wherein the amplitude of this rhythm may be reduced, or the phase of the pattern may be delayed or advanced, or the period of the pattern may be shortened or lengthened, or fragmentation of the pattern may occur.

With ISWRD, a major sleep period is less well articulated, and in some embodiments, sleep is fragmented into, for example, at least three or four periods during a 24-hour day. In some embodiments, the sleep is fragmented into, for example, at least three periods during a 24-hour day. In some embodiments, the sleep is fragmented into, for example, at least four periods during a 24-hour day. The number of sleep fragmentations may vary from day to day. See, e.g., Diagnostic and Statistical Manual of Mental Disorders, Fifth Edition, 2013, 394-95. This disorder is one of the circadian rhythm sleep disorders, and is different from general insomnia disorder.

In some embodiments, ISWRD may be due to at least one neurological disorder. In some embodiments, ISWRD may be due to traumatic brain injury. See, e.g., Viola-Saltzman, M. and Watson, N. F., Neurol. Clin., 2012, 30, 1299-1312 (describing a potential link between traumatic brain injury and irregular sleep-wake rhythm disorder). In some embodiments, ISWRD may be due to at least one neurodevelopmental disorder. See, e.g., Abbott, S. M. and Zee, P. C., Sleep Med. Clin., 2015, 10, 517-22. In some embodiments, the at least one neurodevelopmental disorder may be selected from Angelman Syndrome, autism, mental retardation, and Down Syndrome. See id. In some embodiments, the at least one neurodevelopmental disorder may be Angelman Syndrome. In some embodiments, the at least one neurodevelopmental disorder may be autism. In some embodiments, the at least one neurodevelopmental disorder may be mental retardation. In some embodiments, the at least one neurodevelopmental disorder may be Down Syndrome.

In some embodiments, ISWRD may be due to dementia. In some embodiments, ISWRD may be due to Parkinson's Disease. In some embodiments, ISWRD may be due to dementia due to Alzheimer's Disease. See, e.g., Vitiello, M. V. and Zee, P. C., Sleep Med. Clin., 2009, 4, 213-18. In some embodiments, ISWRD may be due to dementia due to related disorders. See, e.g., id. In some embodiments, ISWRD may be due to dementia due to mild cognitive impairment, e.g., traumatic brain injury. See, e.g., id.

According to the DSM-V, Circadian Rhythm Sleep-Wake Disorders of the Irregular Sleep-Wake Type, are described as “[a] temporally disorganized sleep-wake pattern, such that the timing of sleep and wake periods is available through the 24-hour period.” See, e.g., Diagnostic and Statistical Manual of Mental Disorders, Fifth Edition, 2013, 390.

As used herein, the term “at least one circadian rhythm sleep disorder” means at least one condition chosen from sleep disruption, sleep-wake fragmentation, and circadian rhythm sleep disorder.

As used herein, the term “sleep disruption,” also known as “interrupted sleep,” “divided sleep,” “fragmented sleep,” and “segmented sleep,” among other terms, means a sleep pattern where nighttime sleep is punctuated by one or more periods of wakefulness and daytime is punctuated by one or more periods of sleep. Sleep disruption may be assessed by, for example, wake time after sleep onset (WASO), sleep efficiency, duration of awakenings, and/or number of awakenings (NAW) and sleep fragmentation index (SFI).

As used herein, the term “sundowning” means disorientation beginning at dusk and continuing throughout the night.

As used herein, the term “wake efficiency” refers to the amount of time awake/time out of bed, and includes the number and duration or planned and spontaneous sleep bouts.

As used herein, the term “sleep-wake fragmentation”, also known as “fragmented sleep and wake,” is a disorder characterized by a nocturnal sleep period punctuated by one or more periods of wakefulness and which may be shorter in duration than the subject slept prior to the onset of any of the causes of the sleep disorders. The time spent sleeping during the nighttime sleep period, at any sleep stage, is less continuous than normal. Daytime wakefulness is punctuated with irregular napping.

As used herein, the term “circadian rhythm sleep disorders” is a family of disorders that affect, among other bodily processes, the timing of sleep. A circadian rhythm sleep disorder may be characterized by at least one disruption or disturbance chosen from the following:
A. A persistent or recurrent pattern of sleep disruption that is primarily due to an alteration of the circadian system or to a misalignment between the endogenous circadian rhythm and the sleep-wake schedule required by an individual's physical environment or social or professional schedule;
B. The sleep disruption leads to an excessive sleepiness or insomnia, or both; and
C. The sleep disturbance causes clinically significant distress or impairment in social, occupational, and other important areas of functioning.

### See e.g., id. at 390-98.

As used herein, the terms “stabilization of one or more circadian rhythms,” “stabilizing one or more circadian rhythms,” and “stabilizing a circadian rhythm” mean no increase in one or more disturbances and/or disruptions associated with a circadian rhythm sleep disorder.

As used herein, the terms “improving one or more circadian rhythms,” “improvement of one or more circadian rhythms,” and “improvement of a circadian rhythm” means a reduction in one or more disturbances and/or disruptions associated with a circadian rhythm sleep disorder.

As used herein, the term “dementia” refers to a neurocognitive disorder. See, e.g., Diagnostic and Statistical Manual of Mental Disorders, Fifth Edition, 2013, 602-14; Albert, M. S. et al., Alzheimer's & Dementia, 2011, 7, 271-72; McKhann, G. M., et al., Alzheimer's & Dementia, 2011, 7, 265; Dubois, B., et al., Lancet Neurol., 2014, 13, 614-29.

As used herein, the term “mild cognitive impairment due to Alzheimer's disease” means a slight but noticeable and measurable decline in cognitive abilities, e.g., memory and thinking skills, due to Alzheimer's disease, and is discussed in, e.g., Diagnostic and Statistical Manual of Mental Disorders, Fifth Edition, 2013, 602-14; Albert, M. S. et al., Alzheimer's & Dementia, 2011, 7, 271-72; McKhann, G. M., et al., Alzheimer's & Dementia, 2011, 7, 265; Dubois, B., et al., Lancet Neurol., 2014, 13, 614-29.

As used herein, the terms “clinical stabilization in mild cognitive impairment due to Alzheimer's disease” and “clinical stabilization in dementia due to Alzheimer's disease and/or other neurodegenerative disorders” mean stabilization or no or insignificant decline in at least one domain chosen from cognitive, functional, and behavioral, including but not limited to mood, activities of daily living, agitation, restlessness, etc.

As used herein, the term “clinical stabilization” in ISWRD means stabilization or no or insignificant decline in at least one domain chosen from sleep-wake fragmentation, amplitudes of the sleep-wake and alertness rhythms, the sleep-wake pattern from day to day, sleep efficiency, wake efficiency, etc.

As used herein, the term “clinical improvement” in ISWRD means improvement in at least one domain chosen from sleep-wake fragmentation, amplitudes of the sleep-wake and alertness rhythms, the sleep-wake pattern from day to day, sleep efficiency, wake efficiency, etc.

As used herein, the term “clinical reduction in rate of decline” in ISWRD means a decrease in the rate of decline in at least one domain chosen from at least one domain of sleep-wake fragmentation, amplitudes of the sleep-wake and alertness rhythms, the sleep-wake pattern from day to day, sleep efficiency, wake efficiency, etc.

As used herein, the term “sleep time” refers to the time that a subject spends asleep. Sleep time may be continuous or discontinuous.

As used herein, the term “sleep efficiency” refers to the total sleep time a subject obtains during his or her time in bed. Sleep efficiency is measured by the equation: sleep efficiency=100%*(total sleep time/total time in bed).

As used herein, a “decrease in daytime sleepiness” means an increase and/or stabilization in the percentage of time spent awake during the daytime.

As used herein, a “decrease in daytime napping” means a decrease in the number and duration of daytime napping (planned and unplanned).

As used herein, an “improvement in daytime alertness” means stabilization, improvement, and/or slower rate of decline in assessments of alertness.

As used herein, a “decrease in behavioral disturbances” means stabilization, improvement, and/or slower rate of decline in assessments of agitation and/or behavioral disturbance.

Subjective and objective determinations, for example, actigraphy, of the aforementioned circadian rhythm sleep disorders and quality are known in the art. In some embodiments, the at least one circadian rhythm sleep disorder is determined by subjective measurements, such as, for example, asking the subject, maintaining a sleep diary, or assessment via a standardized questionnaire regarding how restorative and undisturbed sleep has been (e.g., Pittsburgh Sleep Quality Index (Buysse et al., Psychiatry Research, 1989, 28, 193-213)). In some embodiments, the at least one circadian rhythm sleep disorder is determined by observing the subject, such as, for example, observing how long it takes the subject to fall asleep, how many times the subject wakes up during the night, how agitated the subject is (e.g., physical or verbal agitation), how aggressive the subject's behavior is, how disoriented the subject is upon awakening, etc. In some embodiments, the at least one circadian rhythm sleep disorder is determined using polysomnography. Polysomnography is the monitoring of multiple electrophysiological parameters during sleep and generally includes measurement of EEG activity, electro-oculographic activity and electromyographic activity, as well as other measurements. These results, along with observations, can measure not only sleep latency (the amount of time required to fall asleep), but also sleep continuity (overall balance of sleep and wakefulness, or the percentage of time spent asleep or the amount of time spent awake after sleep onset) which may be an indication of the quality of sleep. Actigraphic measurement techniques may be used as well. Actigraphy is the objective measurement of motor activity in a subject using a device that is worn on the body, generally the non-dominant wrist.

Dosage forms of the present disclosure contain lemborexant in a therapeutically effective amount for treatment of, e.g., ISWRD when administered in accordance with the teachings of the present disclosure. The unit dose of the effective amount in a dosage form is from 0.5 mg to 100 mg, from 2 mg to 75 mg, from 2 mg to 70 mg, from 2 mg to 65 mg, from 2 mg to 60 mg, from 2 mg to 55 mg, from 2 mg to 50 mg, from 2 mg to 45 mg, from 2 mg to 40 mg, from 2 mg to 35 mg, from 2 mg to 30 mg, from 2 mg to 25 mg, from 2 mg to 20 mg, from 1 mg to 15 mg, from 2 mg to 15 mg, or chosen from 2 mg, 2.5 mg, 4 mg, 5 mg, 8 mg, 10 mg, or 15 mg. The unit dose is not limited by the type of the dosage form or the number of dosage forms for single dose. In some embodiments, the unit dose may be 2.5 mg. In some embodiments, the unit dose may be 5 mg. In some embodiments, the unit dose may be 10 mg. In some embodiments, the unit dose may be 15 mg.

In some embodiments, a dosage form of the present disclosure may constitute one or more pharmaceutical compositions comprising lemborexant together with pharmaceutically acceptable excipients.

As used herein, the term “composition” used herein includes a product comprising a particular ingredient in a particular amount and any product directly or indirectly brought about by the combination of particular ingredients in particular amounts. Such a term related to the pharmaceutical composition is intended to include a product comprising an active ingredient and an inert ingredient constituting a carrier and include every product directly or indirectly brought about by the combination, complexation or aggregation of any two or more ingredients or the dissociation, other kinds of reactions or interaction of one or more ingredients. Thus, the pharmaceutical composition of the present disclosure includes every composition prepared by mixing the compound of the present disclosure with a pharmaceutically acceptable carrier.

As used herein, the term “pharmaceutically acceptable” means that a carrier, diluent, excipient, or vehicle is compatible with other components of a formulation and is nontoxic to a subject.

Solid dosage forms of the present disclosure include capsules, granules, lozenges, pellets, pills, powders, suspensions, and tablets.

The pharmaceutical compositions of the present disclosure may be prepared using standard techniques and manufacturing processes generally known in the art. See, e.g., the monograph of Japanese Pharmacopoeia, 16^{th} Edition; and Pharmaceutical Dosage Forms of U.S. Pharmacopoeia-NF, Chapter 1151.

In some embodiments, pharmaceutical compositions comprise lemborexant. In some embodiments, pharmaceutical compositions further comprise at least one additional component chosen from pharmaceutically acceptable carriers, pharmaceutically acceptable vehicles, and pharmaceutically acceptable excipients.

In some embodiments, the at least one additional component in the pharmaceutical compositions is chosen depending upon the route of administration for which the pharmaceutical composition is intended. Non-limiting examples of suitable routes of administration for which the pharmaceutical composition may be used include parenteral, oral, inhalation spray, topical, rectal, nasal, buccal, vaginal and implanted reservoir administration. The term “parenteral” as used herein includes subcutaneous, intravenous, intramuscular, intra-articular, intra-synovial, intrasternal, intracisternal, intrathecal, intrahepatic, intralesional and intracranial injection or infusion techniques. In some embodiments, the mode of administration is chosen from intravenous, oral, subcutaneous, and intramuscular administration. Sterile injectable forms of the compositions of this disclosure may be, for example, aqueous or oleaginous suspension. These suspensions may be formulated according to techniques known in the art using suitable dispersing or wetting agents and suspending agents known in the art. The sterile injectable preparation may also be a sterile injectable solution or suspension in a nontoxic parenterally acceptable diluent or solvent, for example, as a solution in 1,3-butanediol. Non-limiting examples of vehicles and solvents that may be employed include water, Ringer's solution, and isotonic sodium chloride solution. In addition, sterile, fixed oils may be employed as a solvent and/or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, and/or other dosage forms, may also be used for the purposes of formulation.

For oral administration, lemborexant, may be provided in an acceptable oral dosage form, including, but not limited to, capsules, tablets, oral disintegrating tablet, sprinkles, and other oral formulations that would be easy to swallow. In some embodiments, lemborexant is provided in the form of tablet or capsules. In some embodiments, lemborexant is provided in the form of crushable tablets. In the case of tablets for oral use, carriers commonly used include lactose and cornstarch. Lubricating agents, such as magnesium stearate, may also be added. For oral administration in a capsule form, useful diluents include lactose and dried cornstarch. When aqueous suspensions are required for oral use, the active ingredient is combined with an emulsifying and/or suspending agent. If desired, certain sweetening, flavoring or coloring agents may also be added.

In some embodiments, the methods disclosed herein comprise administering orally a dosage form comprising lemborexant to the subject. In some embodiments, the methods disclosed herein comprise administering orally a dosage form comprising 5 mg of lemborexant to the subject. In some embodiments, the methods disclosed herein comprise administering orally a dosage form comprising 10 mg of lemborexant to the subject.

In some embodiments, the methods disclosed herein comprise administering orally a dosage form comprising 2.5 mg to 15 mg of lemborexant to the subject no more than once per night, immediately before going to bed, with at least 7 hours remaining before the planned time of awakening, wherein the dose may be increased up to 15 mg, such as up to 5 mg or 10 mg, of lemborexant or an equivalent dose of a pharmaceutically acceptable salt thereof based on clinical response and tolerability.

In some embodiments, the methods disclosed herein comprise administering orally a dosage form comprising 5 mg to 10 mg of lemborexant to the subject no more than once per night, immediately before going to bed, with at least 7 hours remaining before the planned time of awakening, wherein the dose may be increased up to 15 mg of lemborexant or an equivalent dose of a pharmaceutically acceptable salt thereof based on clinical response and tolerability.

In some embodiments, the methods disclosed herein comprise administering orally a dosage form comprising 5 mg of lemborexant to the subject no more than once per night, immediately before going to bed, with at least 7 hours remaining before the planned time of awakening, wherein the dose may be increased up to 10 mg of lemborexant or an equivalent dose of a pharmaceutically acceptable salt thereof based on clinical response and tolerability. In some embodiments, a 5 mg dose may be increased up to 10 mg or up to 15 mg of lemborexant or an equivalent dose of a pharmaceutically acceptable salt thereof based on clinical response and tolerability. In some embodiments, a 10 mg dose may be increased up to 15 mg of lemborexant or an equivalent dose of a pharmaceutically acceptable salt thereof based on clinical response and tolerability. In order that the disclosure described herein may be more fully understood, the following examples are set forth. It should be understood that these examples are for illustrative purposes only and are not to be construed as limiting this disclosure in any manner.

### EXAMPLES

### Example 1

The recommended dose of lemborexant is 5 mg taken no more than once per night, immediately before going to bed, with at least 7 hours remaining before the planned time of awakening. Dosage may be increased to 10 mg based on clinical response and tolerability.

### Example 2. Treatment of Subjects with ISWRD

Subjects, both male and female with ages ranging from 60 to 90 years old, who complain of disrupted sleep or multiple awakenings at night along with frequent periods of falling asleep during the day may be asked to answer the following questionnaire.

During a medical examination, a clinician will verify that a subject has been diagnosed with ISWRD and/or a related circadian rhythm sleep disorders. This is accomplished by the subject's participation in a medical, psychiatric, and sleep history assessment, and mini-mental state examination (“MMSE”) in addition to assessment of the subject's electrocardiogram, clinical laboratory tests, vital signs, height and weight.

The clinician will determine whether the subject is currently on any medication such as lemborexant, any other medication used to treat ISWRD to be eligible for treatment, or any other medication for any other ailments or diseases.

The clinician will ask the subject and/or the subject's aid/nurse the following questionnaire and determine whether a regimen of lemborexant is appropriate or whether the subject's dose of lemborexant needs to be altered (e.g. increased or decreased).

The questionnaire is divided into a Daytime component and a Nighttime component. The questions in each component are multiple choice type questions. The caregiver picks the most appropriate response to each question from the list of multiple choice answers. For each answer, the subject is given a score from 1 to 5 depending on the type of question . . .

1. A method of treating irregular sleep-wake rhythm disorder and related circadian rhythm sleep disorders associated with neurodegenerative diseases comprising administering lemborexant to a subject who achieves a score of greater than or equal to 46 on ISWRD ObsRO Questionnaire Daytime Component.

2. A method of treating irregular sleep-wake rhythm disorder and related circadian rhythm sleep disorders associated with neurodegenerative diseases comprising administering lemborexant to a subject who achieves a score of greater than or equal to 8 on ISWRD ObsRO Questionnaire Nighttime Component.

3. The method according to claim 1, wherein the subject experiences at least one symptom chosen from confusion, anxiety, agitation, pacing, wandering, mental exhaustion, physical exhaustion, and sundowning.

4. The method according to claim 1, wherein the irregular sleep-wake rhythm disorder is due to at least one neurodevelopmental disorder, at least one neurodegenerative disorder, or a combination thereof.

5. The method according to claim 1, wherein the subject suffers from at least one circadian rhythm sleep disorder in addition to irregular sleep-wake rhythm disorder.

6. A method of continuing treatment of irregular sleep-wake rhythm disorder and related circadian rhythm sleep disorders associated with neurodegenerative diseases comprising administering lemborexant to a subject who achieves a score of greater than or equal to 46 on the ISWRD ObsRO Questionnaire Daytime Component.

7. A method of continuing treatment of irregular sleep-wake rhythm disorder and related circadian rhythm sleep disorders associated with neurodegenerative diseases comprising administering lemborexant to a subject who achieves a score of greater than or equal to 8 on the ISWRD ObsRO Questionnaire Nighttime Component.

8. The method according to claim 6, wherein the subject experiences an increase in sleep efficiency.

9. The method according to claim 6, wherein the subject experiences a decrease in daytime sleepiness and/or daytime napping.

10. The method according to claim 6, wherein the subject experiences an increase in wake efficiency.

11. The method according to claim 6, wherein the subject experiences an increase in daytime alertness.

12. The method according to claim 6, wherein the subject experiences a decrease in behavioral disturbances.

13. The method according to claim 6, wherein the subject experiences a decrease in loss in concentration.

14. The method according to claim 6, wherein the subject experiences an increase in undertaking personal care.

15. The method according to claim 6, wherein the subject experiences an increase in emotional wellbeing.

16. The method according to claim 6, wherein the subject experiences an increase in participating in leisure activities.

17. A method of determining therapeutic benefit in the treatment of irregular sleep-wake rhythm disorder and related circadian rhythm sleep disorders associated with neurodegenerative diseases comprising administering ISWRD ObsRO Questionnaire Daytime Component and/or ISWRD ObsRO Questionnaire Nighttime Component to a subject prior to treatment of irregular sleep-wake rhythm disorder and at least one day following treatment of irregular sleep-wake rhythm disorder.

18. The method according to claim 17, wherein treatment of the subject comprises administration of lemborexant.

19. The method according to claim 17, wherein the subject achieves a score of less than 46 on the ISWRD ObsRO Questionnaire Daytime Component after treatment.

20. The method according to claim 17, wherein the subject achieves a score of greater than or equal to 46 on the ISWRD ObsRO Questionnaire Daytime Component prior to treatment.

21. The method according to claim 17, wherein the subject achieves a score of less than to 8 on the ISWRD ObsRO Questionnaire Nighttime Component after treatment.

22. The method according to claim 17, wherein the subject achieves a score of greater than or equal to 8 on the ISWRD ObsRO Questionnaire Nighttime Component prior to treatment.

23. The method according to claim 1, wherein 5 mg or 10 mg of lemborexant is administered to the subject once per night, immediately before going to bed.

24. The method according to claim 1, wherein 5 mg or 10 mg of lemborexant is administered to the subject once per night, immediately before going to bed, with at least 7 hours remaining before the planned time of awakening.

25. The method according to claim 1, wherein 5 mg of lemborexant is administered to the subject once per night.
